(19) United States
(12) Patent Application Publication
(10) Pub. No.: US 2024/0148303 A1
(43) Pub. Date: May 9, 2024

(54) ACUTE HEALTH EVENT MONITORING AND GUIDANCE

(71) Applicant: Medtronic, Inc., Minneapolis, MN (US)

(72) Inventors: Kevin T. Ousdigian, Shoreview, MN (US); Paul G. Krause, Mahtomedi, MN (US); Ryan D. Wyszynski, Oak Grove, MN (US); Megan Connolly, Buffalo, MN (US); Grant A. Neitzell, Loretto, MN (US); Christopher D. Koch, Minneapolis, MN (US)

(21) Appl. No.: 18/549,227

(22) PCT Filed: Feb. 17, 2022
(86) PCT No.: PCT/US2022/016681

§ 371 Date: Sep. 6, 2023

Related U.S. Application Data
(60) Provisional application No. 63/182,499, filed on Apr. 30, 2021.
(60) Provisional application No. 63/158,189, filed on Mar. 8, 2021.

(51) Int. Cl. A61B 5/29; A61B 5/00; A61B 5/308; A61B 5/346; G16H 20/10; G16H 20/30; G16H 50/20

(52) U.S. Cl. CPC A61B 5/29; A61B 5/308; A61B 5/346; A61B 5/7267; A61B 5/7282; A61B 5/7405; A61B 5/742; G16H 20/10; G16H 20/30; G16H 50/20; A61B 2560/0468

(57) ABSTRACT

Example devices, systems, and techniques are disclosed for providing guidance of a treatment of a patient. An example device includes processing circuitry and memory comprising instructions that, when executed by the processing circuitry, cause the processing circuitry to determine that a device detected an acute health event of a patient or delivery of cardiopulmonary resuscitation to the patient, and analyze sensed patient data in response to the determination. The instructions cause the processing circuitry to provide information for guidance of a treatment of the patient based on the analysis.

### FIELD

This disclosure generally relates to systems including medical devices and, more particularly, to monitoring of patient health using such systems.

### BACKGROUND

A variety of devices are configured to configured to monitor physiological signals of a patient. Such devices include implantable or wearable medical devices, as well as a variety of wearable health or fitness tracking devices. The physiological signals sensed by such devices include as examples, electrocardiogram (ECG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

In some cases, such devices are configured to detect acute health events based on the physiological signals, such as episodes of cardiac arrhythmia, myocardial infarction, stroke, or seizure. Example arrhythmia types include cardiac arrest (e.g., asystole), ventricular tachycardia (VT), and ventricular fibrillation (VF). The devices may store ECG and other physiological signal data collected during a time period including an episode as episode data. Such acute health events are associated with significant rates of death, particularly if not treated quickly.

For example, VF and other malignant tachyarrhythmias are the most commonly identified arrhythmia in sudden cardiac arrest (SCA) patients. If this arrhythmia continues for more than a few seconds, it may result in cardiogenic shock and cessation of effective blood circulation. The survival rate from SCA decreases between 7 and 10 percent for every minute that the patient waits for defibrillation. Consequently, sudden cardiac death (SCD) may result in a matter of minutes.

### SUMMARY

In general, the disclosure describes techniques for providing information to a computing device in response to sensing an indication of an acute health event. The information is indicative of guidance for treatment of the patient experiencing the acute health event. In some examples, the information may be provided to a bystander computing device, an automated cardiopulmonary resuscitation (CPR) machine, and/or care provider computing device.

In one example, a device includes processing circuitry and memory comprising instructions that, when executed by the processing circuitry, cause the processing circuitry to: determine that a device detected an acute health event of a patient or delivery of cardiopulmonary resuscitation (CPR) to the patient; analyze sensed patient data in response to the determination; and provide information for guidance of a treatment of the patient based on the analysis.

In another example, a method includes determining, by processing circuitry, that a device detected an acute health event of a patient or delivery of cardiopulmonary resuscitation (CPR) to the patient; analyzing, by the processing circuitry, sensed patient data in response to the determination; and providing, by the processing circuitry, information for guidance of a treatment of the patient based on the analysis.

In another example, a non-transitory computer-readable storage medium stores instructions that, when executed, cause processing circuitry to: determine that a device detected an acute health event of a patient or delivery of cardiopulmonary resuscitation (CPR) to the patient; analyze sensed patient data in response to the determination; and provide information for guidance of a treatment of the patient based on the analysis.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured detect acute health events of a patient, and to respond to such detections, in accordance with one or more techniques of this disclosure.

FIG. 2 is a block diagram illustrating an example configuration of a patient sensing device that operates in accordance with one or more techniques of the present disclosure.

FIG. 3 is block diagram illustrating an example configuration of a computing device that operates in accordance with one or more techniques of the present disclosure.

FIG. 4 is a block diagram illustrating an example configuration of a health monitoring system that operates in accordance with one or more techniques of the present disclosure.

FIG. 5 is a flow diagram illustrating example guidance techniques of the present disclosure.

Like reference characters refer to like elements throughout the figures and description.

### DETAILED DESCRIPTION

A patient suffering from an acute health event, such as a sudden cardiac arrest (SCA), may need robust cardiopulmonary resuscitation (CPR) and/or early defibrillation which can more than double the chance of survival and reduce impairment that may result from the acute health event. However, bystanders often do not deliver CPR correctly at the right cadence and intensity, as many bystanders have not been trained on how to deliver CPR. Additionally, emergency medical services (EMS) often arrive with little knowledge about the patient and their medical condition. Various devices may include sensors that may be utilized to determine and provide guidance to bystanders or automated CPR devices on properly delivering CPR. Such devices may also be used to determine and provide guidance to EMS and/or medical facilities regarding an automated or machine diagnosis of the acute health event which may enable EMS personnel or medical facility staff to be better prepared to provide treatment to the patient when EMS personnel arrive at a location of the patient or when the patient arrives at the medical facility.

Furthermore, when a patient experiences a stroke, the amount of time taken to provide the proper drug or surgical intervention is key in saving as much brain function (e.g., brain cells) of the patient as possible. It may be desirable to route a stroke patient to a comprehensive stroke facility rather than the nearest medical facility to save precious time in the case the nearest facility does not have the proper equipment or protocols in place to provide optimal care to the patient.

A variety of types of implantable and computing devices are configured detect arrhythmia episodes and other acute health events based on sensed ECGs and, in some cases, other physiological signals. Computing devices that may be used to non-invasively sense and monitor ECGs and other physiological signals include wearable devices with electrodes configured to contact the skin of the patient, such as patches, watches, rings, or necklaces, and other non-contact monitoring devices, such as devices configured to monitor sound, radar, light, images, etc. Such computing devices may facilitate relatively longer-term monitoring of patient health during normal daily activities.

Implantable medical devices (IMDs) also sense and monitor ECGs and other physiological signals, and detect acute health events such as episodes of arrhythmia, cardiac arrest, myocardial infarction, stroke, and seizure. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ Insertable Cardiac Monitor (ICM) or the LINQ II™ ICM, available from Medtronic plc, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

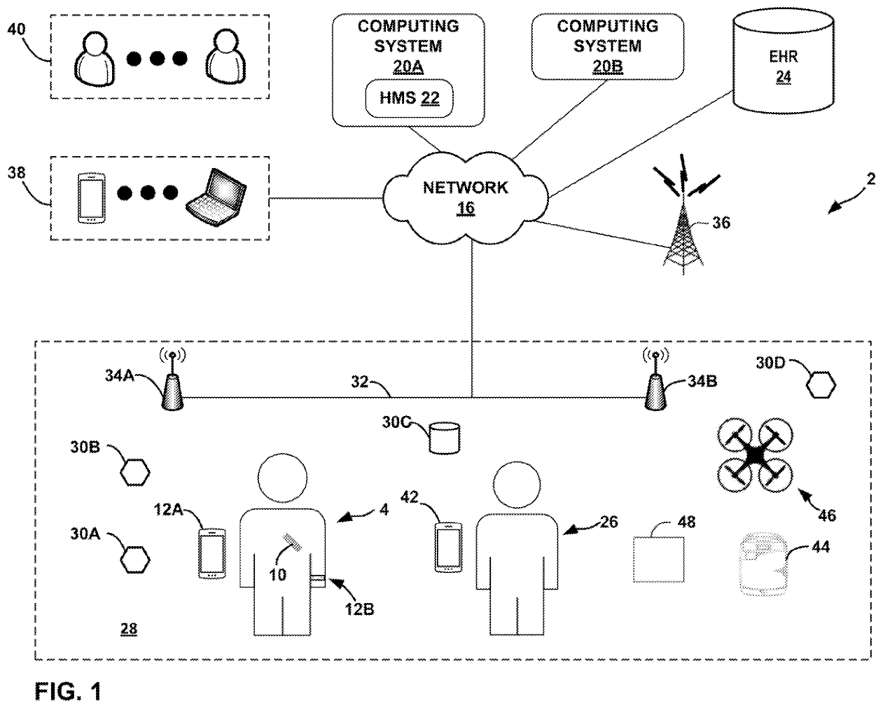

FIG. 1 is a block diagram illustrating an example system 2 configured detect acute health events of a patient 4, and to respond to such detection, in accordance with one or more techniques of this disclosure. As used herein, the terms “detect,” “detection,” and the like may refer to detection of an acute health event presently (at the time the data is collected) being experienced by patient 4, as well as detection based on the data that the condition of patient 4 is such that they have a suprathreshold likelihood of experiencing the event within a particular timeframe, e.g., prediction of the acute health event. The example techniques may be used with one or more patient sensing devices, e.g., IMD 10, which may be in wireless communication with one or more patient computing devices, e.g., patient computing devices 12A and 12B (collectively, “computing devices 12”). Although not illustrated in FIG. 1, IMD 10 include electrodes and other sensors to sense physiological signals of patient 4, and may collect and store sensed physiological data based on the signals and detect episodes based on the data.

IMD 10 may be implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, IMD 10 takes the form of the LINQ™ ICM. Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, or drug pumps. Furthermore, although described primarily in the context of examples including a single implanted patient sensing device, in some examples a system includes one or more patient sensing devices, which may be implanted within patient 4 or external to (e.g., worn by) patient 4.

Computing devices 12 are configured for wireless communication with IMD 10. Computing devices 12 retrieve event data and other sensed physiological data from IMD 10 that was collected and stored by the IMD. In some examples, computing devices 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, or tablet computer. Computing devices 12 may communicate with IMB 10 and each other according to the Bluetooth® or Bluetooth® Low Energy (BLE) protocols, as examples. In some examples, only one of computing devices 12, e.g., computing device 12A, is configured for communication with IMB 10, e.g., due to execution of software (e.g., part of a health monitoring application as described herein) enabling communication and interaction with an IMD.

In some examples, computing device(s) 12, e.g., wearable computing device 12B in the example illustrated by FIG. 1A, may include electrodes and other sensors to sense physiological signals of patient 4, and may collect and store physiological data and detect episodes based on such signals. Computing device 12B may be incorporated into the apparel of patient 14, such as within clothing, shoes, eyeglasses, a watch or wristband, a hat, a ring, a necklace, etc. In some examples, computing device 12B is a smartwatch or other accessory or peripheral for a smartphone computing device 12A.

One or more of computing devices 12 may be configured to communicate with a variety of other devices or systems via a network 16. For example, one or more of computing devices 12 may be configured to communicate with one or more computing systems, e.g., computing systems 20A and 20B (collectively, “computing systems 20”) via network 16. Computing systems 20A and 20B may be respectively managed by manufacturers of IMD 10 and computing devices 12 to, for example, provide cloud storage and analysis of collected data, maintenance and software services, or other networked functionality for their respective devices and users thereof. Computing system 20A may comprise, or may be implemented by, the Medtronic Carelink™ Network, in some examples. In the example illustrated by FIG. 1, computing system 20A implements a health monitoring system (HMS) 22, although in other examples, either of both of computing systems 20 may implement HMS 22. As will be described in greater detail below, HMS 22 facilities detection of acute health events of patient 4 by system 2, and the responses of system 2 to such acute health events.

Computing device(s) 12 may transmit data, including data retrieved from IMD 10, to computing system(s) 20 via network 16. The data may include sensed data, e.g., values of physiological parameters measured by IMD 10 and, in some cases one or more of computing devices 12, data regarding episodes of arrhythmia or other acute health events detected by IMD 10 and computing device(s) 12, and other physiological signals or data recorded by IMD 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing devices 12 to detect acute health events for patient 4. In some examples, HMS 22 provides data from EHR 24 to computing device(s) 12 and/or IMD 10 for storage therein and use as part of their algorithms for detecting acute health events.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, IMB 10 may be configured to detect acute health events of patient 4 based on data sensed by IMD 10 and, in some cases, other data, such as data sensed by computing devices 12A and/or 12B, and data from EHR 24. In response to detection of an acute health event, IMB 10 may wirelessly transmit a message to one or both of computing devices 12A and 12B. The message may indicate that IMD 10 detected an acute health event of the patient. The message may indicate a time that IMD 10 detected the acute health event. The message may include physiological data collected by IMB 10, e.g., data which lead to detection of the acute health event, data prior to detection of the acute health event, and/or real-time or more recent data collected after detection of the acute health event. The physiological data may include values of one or more physiological parameters and/or digitized physiological signals. Some examples of acute health events are cardiac arrest, ventricular fibrillation, ventricular tachycardia, myocardial infarction, pause in heat rhythm (asystole), pulseless electrical activity (PEA), acute respiratory distress syndrome (ARDS), stroke, seizure, fall, anaphylactic shock, or respiratory failure.

In response to the message from IMD 10, computing device(s) 12 may output an alarm that may be visual and/or audible, and configured to immediately attract the attention of patient 4 or any person in environment 28 with patient 4, e.g., a bystander 26. Environment 28 may be a home, office, or place of business, or public venue, as examples. Computing device(s) 12 may also transmit a message to HMS 22 via network 16. The message may include the data received from IMD 10 and, in some cases, additional data collected by computing device(s) 12 or other devices in response to the detection of the acute health event by IMD 10. For example, the message may include a location of patient 4 determined by computing device(s) 12.

Other devices in the environment 28 of patient 4 may also be configured to output alarms or take other actions to attract the attention of patient 4 and, possibly, a bystander 26, or to otherwise facilitate the delivery of care to patient 4. For example, environment 28 may include one or more Internet of Things (IoT) devices, such as IoT devices 30A-30D (collectively “IoT devices 30”) illustrated in the example of FIG. 1. IoT devices 30 may include, as examples, so called “smart” speakers, cameras, lights, locks, thermostats, appliances, actuators, controllers, or any other smart home (or building) devices. In the example of FIG. 1, IoT device 30C is a smart speaker and/or controller, which may include a display. IoT devices 30 may provide audible and/or visual alarms when configured with output devices to do so. As other examples, IoT devices 30 may cause smart lights throughout environment 28 to flash or blink and unlock doors. In some examples, IoT devices 30 that include cameras or other sensors may activate those sensors to collect data regarding patient 4, e.g., for evaluation of the condition of patient 4.

Computing device(s) 12 may be configured to wirelessly communicate with IoT devices 30 to cause IoT devices 30 to take the actions described herein. In some examples, HMS 22 communicates with IoT devices 30 via network 16 to cause IoT devices 30 to take the actions described herein, e.g., in response to receiving the alert message from computing device(s) 12 as described above. In some examples, IMD 10 is configured to communicate wirelessly with one or more of IoT devices 30, e.g., in response to detection of an acute health event when communication with computing devices 12 is unavailable or not preferred. In such examples, IoT device(s) 30 may be configured to provide some or all of the functionality ascribed to computing devices 12 herein.

Environment 28 includes computing facilities, e.g., a local network 32, by which computing devices 12, IoT devices 30, and other devices within environment 28 may communicate via network 16, e.g., with HMS 22. For example, environment 28 may be configured with wireless technology, such as 802.11 wireless networks, 802.15 ZigBee networks, an ultrawideband protocol, near-filed communication, or the like. Environment 28 may include one or more wireless access points, e.g., wireless access points 34A and 34B (collectively, “wireless access points 34”) that provide support for wireless communications throughout environment 28. Additionally or alternatively, e.g., when local network is unavailable, computing devices 12, IoT devices 30, and other devices within environment 28 may be configured to communicate with network 16, e.g., with HMS 22, via a cellular base station 36 and a cellular network.

Computing device(s) 12, and in some examples IoT devices 30, may include input devices and interfaces to allow a user to override the alarm in the event the detection of the acute health event by IMD 10 was false. In some examples, one or more of computing device(s) 12 and IoT device(s) 30 may implement an event assistant. The event assistant may provide a conversational interface for patient 4 and/or bystander 26 to exchange information with the computing device or IoT device. The event assistant may query the user regarding the condition of patient 4 in response to receiving the alert message from IMD 10. Responses from the user may be used to confirm or override detection of the acute health event by IMD 10, or to provide additional information about the acute health event or the condition of patient 4 more generally that may improve the efficacy of the treatment of patient 4. For example, information received by the event assistant may be used to provide an indication of severity or type (differential diagnosis) for the acute health event. The event assistant may use natural language processing and context data to interpret utterances by the user. In some examples, in addition to receiving responses to queries posed by the assistant, the event assistant may be configured to respond to queries posed by the user. For example, patient 4 may indicate that they feel dizzy and ask the event assistant, “how am I doing?”.

In some examples, computing device(s) 12 and/or HMS 22 may implement one or more algorithms to evaluate the sensed physiological data received from IMD 10, and in some cases additional physiological or other data sensed or otherwise collected by the computing device(s) or IoT devices 30, to confirm or override the detection of the acute health event by IMB 10. In some examples, computing device(s) 12 and/or computing system(s) 20 may have greater processing capacity than IMD 10, enabling more complex analysis of the data. In some examples, the computing device(s) 12 and/or HMS 22 may apply the data to a machine learning model or other artificial intelligence developed algorithm, e.g., to determine whether the data is sufficiently indicative of the acute health event.

In examples in which computing device(s) 12 are configured to perform an acute health event confirmation analysis, computing device(s) 12 may transmit alert messages to HMS 22 and/or IoT devices 30 in response to confirming the acute health event. In some examples, computing device(s) 12 may be configured to transmit the alert messages prior to completing the confirmation analysis, and transmit cancellation messages in response to the analysis overriding the detection of the acute health event by IMD 10. HMS 22 may be configured to perform a number of operations in response to receiving an alert message from computing device(s) 12 and/or IoT device(s) 30. HMS 22 may be configured to cancel such operations in response to receiving a cancellation message from computing device(s) 12 and/or IoT device(s) 30. In some examples, the acute health event confirmation analysis may include determining an automated or machine diagnosis of the acute health event. For example, IMB 10, computing device(s) 12, IoT device(s) 30, and/or HMS 22 may utilize sensed physiological data to determine an automated or machine diagnosis of the acute health event.

For example, HMS 22 may be configured to transmit alert messages to one or computing devices 38 associated with one or more care providers 40 via network 16. Care providers may include emergency medical systems (EMS) and hospitals, and may include particular departments within a hospital, such as an emergency department, catheterization lab, or a stroke response department. Computing devices 38 may include smartphones, desktop, laptop, or tablet computers, or workstations associated with such systems or entities, or employees of such systems or entities. The alert messages may include any of the data collected by IMD 10, computing device(s) 12, and IoT device(s) 30, including sensed physiological data, time of the acute health event, location of patient 4, and results of the analysis by IMD 10, computing device(s) 12, IoT device(s) 30, and/or HMS 22. In some examples, the results of the analysis may include an automated or machine diagnosis of the acute health event. The information transmitted from HMS 22 to care providers 40 may improve the timeliness and effectiveness of treatment of the acute health event of patient 4 by care providers 40. In some examples, instead of or in addition to HMS 22 providing an alert message to one or more computing devices 38 associated with an EMS care provider 40, computing device(s) 12 and/or IoT devices 30 may be configured to automatically contact EMS, e.g., autodial 911 (e.g., in the United States or North America to use the telephone system to contact a 911 call center), in response to receiving an alert message from IMD 10. Again, such operations may be cancelled by patient 4, bystander 26, or another user via a user interface of computing device(s) 12 or IoT device(s) 30, or automatically cancelled by computing device(s) 12 based on a confirmatory analysis performed by the computing device(s) overriding the detection of the acute health event by IMD 10.

Similarly, HMS 22 may be configured to transmit an alert message to computing device 42 of bystander 26, which may improve the timeliness and effectiveness of treatment of the acute health event of patient 4 by bystander 26. Computing device 42 may be similar to computing devices 12 and computing devices 38, e.g., a smartphone. In some examples, HMS 22 may determine that bystander 26 is proximate to patient 4 based on a location of patient 4, e.g., received from computing device(s) 12, and a location of computing device 42, e.g., reported to HMS 22 by an application implemented on computing device 42. In some examples, HMS 22 may transmit the alert message to any computing devices 42 in an alert area determined based on the location of patient 4, e.g., by transmitting the alert message to all computing devices in communication with base station 36.

In some examples, the alert message (whether from HMS 22, IMD 10, or computing device(s) 12) to bystander 26 may be configured to assist a layperson in treating patient. For example, the alert message to bystander 26 may include a location (and in some cases a description) of patient 4, the general nature of the acute health event, directions for providing care to patient 4, such as directions for providing cardio-pulmonary resuscitation (CPR), a location of nearby medical equipment for treatment of patient 4, such as an automated external defibrillator (AED) 44 or live vest, and instructions for use of the equipment. In some examples, computing device(s) 12, IoT device(s) 30, and/or computing device 42 may implement an event assistant configured to use video, natural language processing, and/or context data to provide a conversational interface for bystander 26. The assistant may provide bystander 26 with directions for providing care to patient 4 such as instructions on how to provide CPR, and respond to queries from bystander 26 about how to provide care to patient 4.

For example, IMD 10 may sense an indication of an acute health event of patient 4. In response to sensing the indication of the acute health event, IMD 10 may communicatively connect to computing device(s) 12, computing device 42, and/or IoT device(s) 30. In some examples, IMD 10 may provide information for guidance of a treatment of patient 4 to computing device(s) 12, computing device 42, and/or IoT device(s) 30, which in turn may provide guidance to bystander 26 or EMS personnel in environment 28. In other examples, IMD 10 may provide the indication of the acute health event and/or other sensed patient data to computing device(s) 12, computing device 42, and/or IoT device(s) 30 and processing circuitry of computing device(s) 12, computing device 42, and/or IoT device(s) 30 may analyze sensed patient data (e.g., from IMD 10, IoT device(s) 30, and/or drone 46) in response to the determination and provide information for guidance of the treatment of the patient to bystander 26. In some examples, the guidance may be audio and/or visual guidance.

In some examples, the information includes the indication of the acute health event and, potentially, other data sensed by IMD 10. In some examples, based on the information, computing device(s) 12, computing device 42, and/or IoT device(s) 30 may determine the guidance and provide the guidance to bystander 26. In some examples, to determine the guidance, computing device(s) 12, computing device 42, and/or IoT device(s) 30 may communicate with HMS and HMS may determine the guidance and provide the guidance to computing device(s) 12, computing device 42, and/or IoT device(s) 30. In some examples, the information includes the guidance and computing device(s) 12, computing device 42, and/or IoT device(s) 30 may provide the guidance to bystander 26.

Bystander 26, following the guidance, may nevertheless not administer CPR correctly to patient 4, especially if bystander 26 is not trained in administering CPR. IMD 10 may include an accelerometer (not shown in FIG. 1). IMD 10 may use accelerometer signals to sense the CPR delivery and whether the CPR delivery is being performed properly. For example, the accelerometer signals may be indicative of an intensity and/or frequency of chest compressions.

In some examples, the accelerometer signals may be used to determine guidance to change the intensity and/or the frequency of the chest compressions. For example, IMD 10 may provide computing device(s) 12, computing device 42, and/or IoT device(s) 30 with information for guidance of a change in treatment of patient 4. In some examples, the information includes the guidance of a change in treatment. In other examples, computing device(s) 12, computing device 42, and/or IoT device(s) 30 may use the information to determine the guidance of the change in treatment. In some examples, computing device(s) 12, computing device 42, and/or IoT device(s) 30 may communicate with HMS 22 and HMS 22 may determine the guidance of the change in treatment and provide the guidance of the change in treatment to computing device(s) 12, computing device 42, and/or IoT device(s) 30, which may in turn provide the guidance of the change in treatment to bystander 26. In this manner, computing device(s) 12, computing device 42, and/or IoT device(s) 30 may provide real-time guidance to bystander 26 regarding the treatment of patient 4. Because IMD 10 is positioned near the sternum near or just below the level of the heart of patient 4 and is relatively stable within that position, information from accelerometer signals of an accelerometer in IMD 10 may be relatively accurate for determining CPR guidance and diagnosing the hearth rhythm of patient 4 when compared to accelerometers in other devices, such as computing device(s) 12 or computing device 42.

In some examples, a combination of sensors of IMD 10 or external information may be synthesized using an algorithm to determine and provide robust guidance. The algorithm may reside in memory IMD 10, computing device(s) 12, computing device 42, IoT device(s) 30, and/or HMS 22. Processing circuitry may utilize the algorithm to determine useful guidance based on sensor data. This guidance may be used by bystander 26 to deliver higher quality CPR. For example, any of computing device(s) 12, computing device 42, and/or IoT device(s) 30 may output a cadence for use by bystander 26 when providing CPR and provide feedback regarding how well bystander 26 is providing CPR or changes bystander 26 should make to improve the quality of the CPR bystander 26 is providing to patient 4.

In some examples, HMS 22 may mediate bi-directional audio (and in some cases video) communication between care providers 40 and patient 4 or bystander 26. Such communication may allow care providers 40 to evaluate the condition of patient 4, e.g., through communication with patient 4 or bystander 26, or through use of a camera or other sensors of IMD 10, computing device(s) 12, or IoT device(s) 30, in advance of the time they will begin caring for the patient, which may improve the efficacy of care delivered to the patient. Such communication may also allow the care providers to instruct bystander 26 regarding first responder treatment of patient 4.

In some examples, rather than instruct bystander 26 to administer CPR, computing device(s) 12, computing device 42, IoT device(s) 30, drone 46, HMS 22, or care providers 40 may instruct bystander 26 to retrieve automated CPR device 48 and place automated CPR device on patient 4. In some examples, automated CPR device may be configured to deliver CPR to patient 4. In some examples, IMD 10 may sense the administration of CPR by automated CPR device 48. IMD 10 may communicatively connect to automated CPR device 48 and provide information for guidance of a treatment (e.g., CPR) of the patient to automated CPR device 48, which may be an example of a computing device. For example, IMD 10 may determine automated CPR device 48 is not providing optimal CPR and may control to administration of CPR by automated CPR device 48. For example, IMD 10 may closed-loop control automated CPR device 48 to provide better CPR based on accelerometer, and possibly, other data sensed by IMD 10. In other examples, rather than control administration of CPR by automated CPR device 48, IMD 10 may provide accelerometer data to automated CPR device 48 and automated CPR device 48 may determine that automated CPR device 48 is not providing optimal CPR and change the administration of CPR based on the accelerometer data. In another example, IMD 10 may provide accelerometer data to computing device(s) 12 or computing device 42 and computing device(s) 12 or computing device 42 may determine that automated CPR device 48 is not providing optimal CPR. In this example, computing device(s) 12 or computing device 42 may control automated CPR device 48 to change the administration of CPR based on the accelerometer data

In some examples, any of computing device(s) 12, computing device 42, and/or IoT device(s) 30 may further communicatively connect with any of computing devices 38 or any of computing devices 38 may further communicatively connect with any of computing device(s) 12, computing device 42, and/or IoT device(s) 30. As such, care providers 40 may further provide instructions or guidance to bystander 26 on treatment of patient 4, for example, through a live stream session or phone call.

In some examples, IMD 10 may detect that a shock or care was or is delivered and the guidance may be changed based on the detected shock or care. In some examples, the guidance may include a list of do not do's associated with treating a patient experiencing an acute health event, such as sudden cardiac arrest. In some examples, the guidance may include instructions not to give tissue plasminogen activator or an intravenous treatment to patient 4 if patient 4 is a hemorrhagic stroke patient, or if accelerometer signals indicated a big impact. In some examples, the guidance may include not administering care, for example, if patient has a “do not resuscitate” order known to care providers 40.

In some examples, HMS 22 may control dispatch of a drone 46 to environment 28, or a location near environment 28 or patient 4. Drone 46 may be a robot and/or unmanned aerial vehicle (UAV). Drone 46 may be equipped with a number of sensors and/or actuators to perform a number of operations. For example, drone 46 may include a camera or other sensors to navigate to its intended location, identify patient 4 and, in some cases, bystander 26, and to evaluate a condition of patient, such as taking an ECG or measuring a pulse. In some examples, drone 46 may act as an AED by touching two parts of the body of patient 4 with extendable arms having electrodes. In some examples, drone 46 may include user interface devices to communicate with patient 4 and/or bystander 26. In some examples, drone 46 may provide directions to bystander 26, to the location of patient 4 and regarding how to provide first responder care, such as CPR, to patient 4. In some examples, drone 46 may carry medical equipment, e.g., AED 44, and/or medication to the location of patient 4.

In some examples, data sensed by IMB 10 may be utilized to provide information for guidance of treatment to computing device(s) 38 of an EMS while EMS personnel are traveling to the location of patient 4 or to emergency room personnel at a medical facility. This may facilitate a quicker medical intervention by EMS personnel when they arrive at the location of patient 4 and when patient 4 arrives at the medical facility, as EMS personnel and emergency room personnel may have a better understanding on which therapy to use when they first see patient 4. For example, IMB 10 may sense an indication of an acute health event of patient 4. IMD 10 may, based on the sensing of the indication of the cardiac event, automatically record an ECG of patient 4. IMB 10 may provide information for guidance of a treatment of patient 4 to computing device(s) 38 or HMS 22. The information may include the ECG. In some examples, computing device(s) 38 or HMS 22 may employ an algorithm, which may be based on machine learning, to analyze the information and determine an automated or machine diagnosis. For example, IMD 10 may sense an asystole. The information may include an indication of the sensed asystole. Computing device(s) 38 or HMS 22 may analyze the information to determine whether the asystole is a true long duration asystole. In some examples, the sensed asystole may be classified as a true long duration asystole by IMB 10, computing device(s) 38 or HMS 22. In some examples, the ECG, classification, or automated or machine diagnosis, may be provided to EMS personnel and/or emergency room personnel, via computing device(s) 38, so that the EMS personnel can have the appropriate therapy ready for patient 4 when they arrive at the location of patient 4 and emergency room personnel can have the appropriate therapy ready for patient 4 when patient 4 arrives at the medical facility. For example, patient 4 may need CPR and Epinephrine as soon as possible and if EMS personnel were not in possession of the ECG, classification, or automated or machine diagnosis, EMS personnel would lose valuable time assessing the condition of patient 4 upon arrival at the location of patient 4. In contrast, if IMB 10 detects a tachyarrhythmia and IMB 10, computing device(s) 38, or HMS 22, determines the rhythm is a rapid, sustained true ventricular arrhythmia, the therapy should be to shock the patient using AED 44 to return the heart to a sinus rhythm. EMS personnel can administer faster therapy by understanding the health situation of patient 4 before they arrive at the location of patient 4.

In some examples, computing device(s) 38 associated with the EMS personnel and/or emergency room personnel may include a smart phone, a smart watch, a tablet, a laptop computer, a desktop computer, or the like. Guidance may be provided to the EMS personnel and/or the emergency room personnel via computing device(s) 38. For example, the guidance may include an indication of what has happened to patient 4 and facilitate the proper treatment of patient 4 for the health situation of patient 4.

For example, any of, or combination of IMD 10, computing device(s) 38, or HMS 22 may be configured to detect the difference between an ischemic or hemorrhagic stroke and provide an automated or machine diagnosis to the EMS personnel and/or emergency room personnel. This automated or machine diagnosis may speed up the time to intervention and/or speed up a change in treatment as EMS personnel and emergency room personnel would have a better idea of the health situation of patient 4 than they otherwise would. Most diagnostic tools (e.g., computerized tomography machines, magnetic resonance imaging machines, blood test laboratories) are located in a medical facility and a diagnosis using such diagnostic tools takes time. The techniques of this disclosure may inform care providers to prepare for needed treatment, which may speed care provided to patient 4 and improve the chances of the best possible outcomes for patient 4. For example, if the automated or machine diagnosis was of an ischemic stroke, care providers may treat patient 4 with an intravenous injection of tissue plasminogen activator, whereas if the automated or machine diagnosis was of a hemorrhagic stroke, care providers may implant a stent.

In some examples, IMD 10 may be configured to sense data that may be utilized to determine an automated or machine diagnosis of a variety of acute health events, such as acute myocardial infarction, lethal arrythmias, stroke, respiratory failure and other emergency medical conditions. IMD 10, computing device(s) 12, IoT device(s) 30, and/or HMS 22 may also be configured to provide relevant information regarding the medical state of patient 4, diagnosis of patient 4, and pertinent posttrial data (e.g., link to acute care guidelines), to care providers 40 in an effort to better inform care needed by patient 4, both ambulatory, as well as in a medical facility.

Certain acute health events, such as stroke, may require a comprehensive workup to enable care providers to identify the problem with patient 4 and provide the proper care in a timely manner. Not all medical facilities have the necessary equipment to perform these tasks. When time is of the greatest importance, there is a need to find the “best” match of a hospital rather than the closest hospital. For example, IMD 10 may sense an indication of an acute health event of patient 4. Upon sensing of the indication of the acute health event or automated or machine diagnosis of the acute health event, such as a stroke, a computing device such as one of computing device(s) 38, or HMS 22 may use the location of the patient 4, and a database of tiered medical facilities (which may be stored in computing device(s) 38, or HMS 22) to determine the best match for patient 4 based on the indication of the acute health event or the automated or machine diagnosis. Computing device(s) 38 may notify EMS personnel of the likely condition of patient 4 and the need for a specific medical facility routing (e.g., routing to the best match) when or before EMS personnel arrive at the location of patient 4. In some examples, computing device(s) 38 or HMS 22 may send an alert to the selected medical facility including information relating to the sensed indication of the acute health event and/or the automated or machine diagnosis, to facilitate the preparation of equipment, personnel, or facilities at the selected medical facility for arrival of patient 4, such that treatment may begin as soon as possible upon arrival at the selected medical facility.

In the case of a stroke, an identification of a “last known well” time may be important information that EMS may use to determine if key drugs can be delivered. In some examples, IMB 10 may be configured to determine a last known well time of patient 4 and provide the last known well time to computing device(s) 38. In other examples, computing device(s) 38 or HMS 22 may be configured to determine the last known well time of patient 4 based on information provided from IMD 10. In the case that HMS 22 determines the last known well time of patient 4, HMS 22 may be configured to provide the last known well time to computing device(s) 38.

IMD 10, computing device(s) 38, and/or HMS 22 may be configured to determine the automated or machine diagnosis using different sensor data based on the type of indication of the acute health event sensed by IMB 10. For example, IMB 10, computing device(s) 38, and/or HMS 22 may determine the automated or machine diagnosis using an ECG if the indication of the acute health event is associated with the heart of patient 4, an EEG if the indication is associated with brain activity, an accelerometer, ECG, and/or impedance measurement if the indication is associated with respiratory distress, or oxygen saturation if the indication is associated with profusion issues, etc.

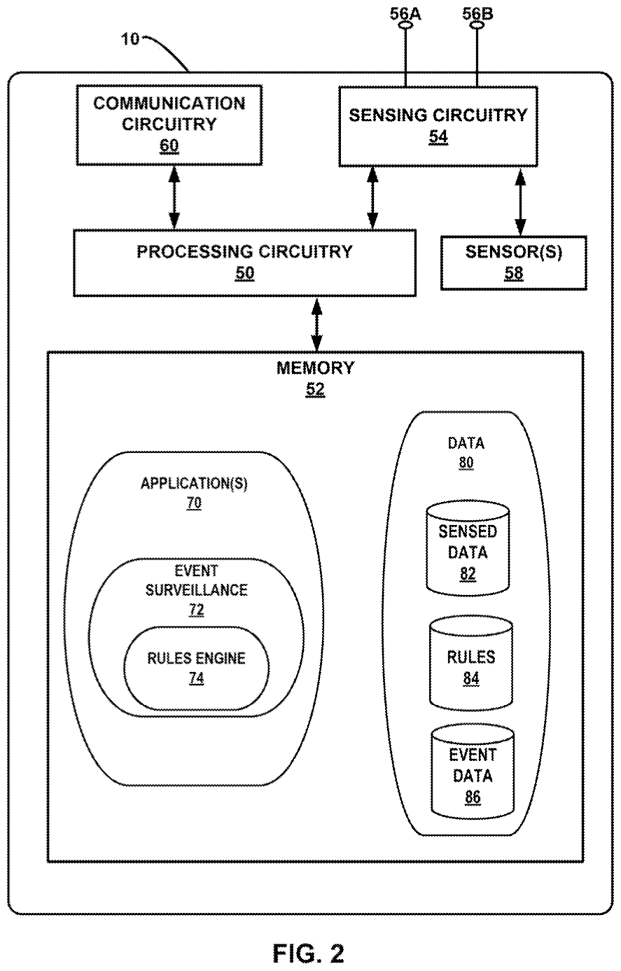

FIG. 2 is a block diagram illustrating an example configuration of IMB 10 of FIG. 1. As shown in FIG. 2, IMB 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 52 includes computer-readable instructions that, when executed by processing circuitry 50, cause IMD 10 and processing circuitry 50 to perform various functions attributed herein to IMB 10 and processing circuitry 50. Memory 52 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 52 as episode data for the detected arrhythmia episode.

In some examples, sensing circuitry 54 measures impedance, e.g., of tissue proximate to IMB 10, via electrodes 56. The measured impedance may vary based on respiration and a degree of perfusion or edema. Processing circuitry 50 may determine physiological data relating to respiration, perfusion, and/or edema based on the measured impedance.

In some examples, IMD 10 includes sensing circuitry 54, such as one or more accelerometers, microphones, optical sensors, temperature sensors, and/or pressure sensors. In some examples, sensing circuitry 54 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52.

Memory 52 may store applications 70 executable by processing circuitry 50, and data 80. Applications 70 may include an acute health event surveillance application 72. Processing circuitry 50 may execute event surveillance application 72 to detect an acute health event of patient 4 based on combination of one or more of the types of physiological data described herein, which may be stored as sensed data 82. In some examples, sensed data 82 may additionally include data sensed by other devices, e.g., computing device(s) 12, and received via communication circuitry 60. Event surveillance application 72 may be configured with a rules engine 74. Rules engine 74 may apply rules 84 to sensed data 82. Rules 84 may include one or more models, algorithms, decision trees, and/or thresholds. In some cases, rules 84 may be developed based on machine learning.

As examples, event surveillance application 72 may detect a cardiac arrest, a ventricular fibrillation, a ventricular tachycardia, a cardiac pause of asystole, pulseless electrical activity (PEA), or a myocardial infarction based on an ECG and/or other physiological data indicating the electrical or mechanical activity of heart 6 of patient 4 (FIG. 1). In some examples, event surveillance application 72 may detect stroke based on such cardiac activity data. In some examples, sensing circuitry 54 may detect brain activity data, e.g., an electroencephalogram (EEG) via electrodes 56, and event surveillance application 72 may detect stroke or a seizure based on the brain activity alone, or in combination with cardiac activity data or other physiological data. In some examples, event surveillance application 72 detects whether the patient has fallen based on data from an accelerometer alone, or in combination with other physiological data. When event surveillance application 72 detects an acute health event, event surveillance application 72 may store the sensed data 82 that lead to the detection (and in some cases a window of data preceding and/or following the detection) as event data 86.

In some examples, in response to detection of an acute health event, processing circuitry 50 transmits, via communication circuitry 60, event data 86 for the event to computing device(s) 12 (FIG. 1). This transmission may be included in a message indicating the acute health event, as described herein. Transmission of the message may occur on an ad hoc basis and as quickly as possible. Communication circuitry 60 may include any suitable hardware, firmware, software, or any combination thereof for wirelessly communicating with another device, such as computing devices 12 and/or IoT devices 30.

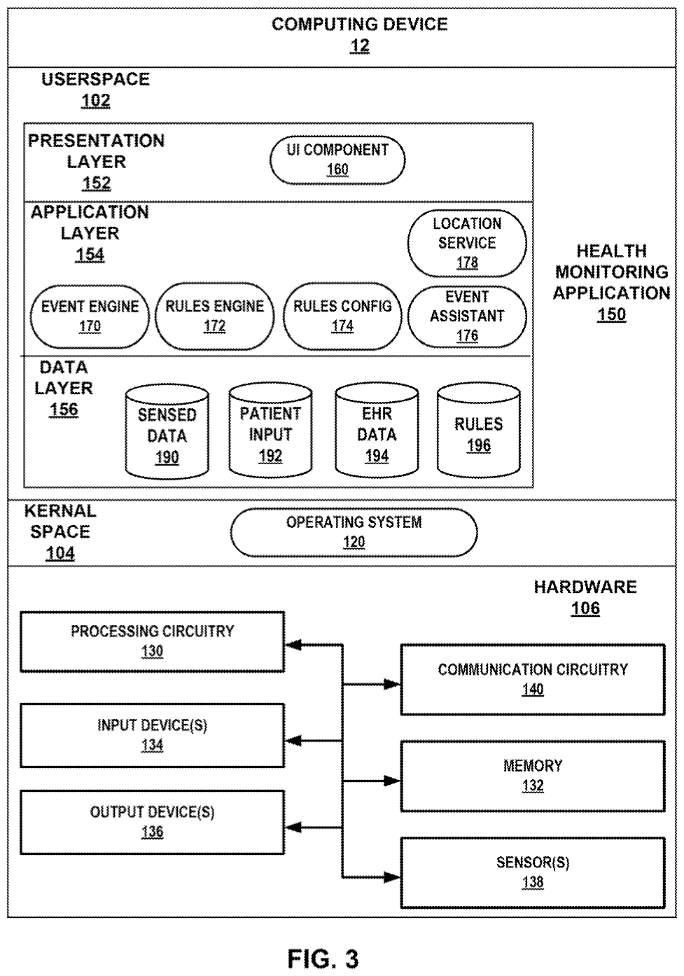

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a laptop, a tablet computer, a personal digital assistant (PDA), a smartwatch or other wearable computing device. In some examples, computing device 42, IoT devices 30, and automated CPR device 48 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, sensing circuitry 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, audio, and visual output. Output devices 136 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

Sensing circuitry 138 of computing device 12 may sense physiological parameters or signals of patient 4. Sensor(s) 138 may include electrodes, 3-axis accelerometers, an optical sensor, impedance sensors, temperature sensors, pressure sensors, heart sounds sensors, and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 3, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, an event engine 170, rules engine 172, rules configuration component 174, event assistant 176, and location service 178. Event engine 170 may be responsive to receipt of an alert transmission from IMB 10 indicating that IMD 10 detected an acute health event. Event engine 170 may control performance of any of the operations in response to detection of an acute health event ascribed herein to computing device 12, such as activating an alarm, transmitting alert messages to HMS 22, controlling IoT devices 30, and analyzing data to confirm or override the detection of the acute health event by IMD 10.

Rules engine 172 analyzes sensed data 190, and in some examples, patient input 192 and/or EHR data 194, to determine whether there is a sufficient likelihood that patient 4 is experiencing the acute health event detected by IMB 10. Sensed data 190 may include data received from IMD 10 as part of the alert transmission, additional data transmitted from IMD 10, e.g., in “real-time,” and physiological and other data related to the condition of patient 4 collected by computing device(s) 12 and/or IoT devices 30. As examples sensed data 190 from computing device(s) 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Patient input 192 may include responses to queries posed by health monitoring application 150 regarding the condition of patient 4, input by patient 4 or another user, such as bystander 26. The queries and responses may occur responsive to the detection of the event by IMB 10, or may have occurred prior to the detection, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition or treatments of patient 4 described above. EHR data 194 may relate to history of cardiac arrest, tachyarrhythmia, myocardial infarction, stroke, seizure, chronic obstructive pulmonary disease (COPD), renal dysfunction, or hypertension, history of procedures, such as ablation or cardioversion, and healthcare utilization. EHR data 194 may also include demographic and other information of patient 4, such as age, gender, height, weight, and BMI.

Rules engine 172 may apply rules 196 to the data. Rules 196 may include one or more models, algorithms, decision trees, and/or thresholds. In some cases, rules 196 may be developed based on machine learning. In some examples, rules 196 and the operation of rules engine 172 may provide a more complex analysis of the sensed data received from IMD 10, than is provided by rules engine 74 and rules 84. In some examples, rules 196 include one or more models developed by machine learning, and rules engine 172 applies feature vectors derived from the data to the model(s).

Rules configuration component 174 may be configured to modify rules 196 (and in some examples rules 84) based on feedback indicating whether the detections and confirmations of acute health events by IMB 10 and computing device 12 were accurate. The feedback may be received from patient 4, or from care providers 40 and/or EHR 24 via HMS 22. In some examples, rules configuration component 174 may utilize the data sets from true and false detections and confirmations for supervised machine learning to further train models included as part of rules 196.

As discussed above, event assistant 176 may provide a conversational interface for patient 4 and/or bystander 26 to exchange information with computing device 12. Event assistant 176 may query the user regarding the condition of patient 4 in response to receiving the alert message from IMB 10. Responses from the user may be included as patient input 192. Event assistant 176 may use natural language processing and context data to interpret utterances by the user. In some examples, in addition to receiving responses to queries posed by the assistant, event assistant 176 may be configured to respond to queries posed by the user. In some examples, event assistant 176 may provide directions to and respond to queries regarding treatment of patient 4 from patient 4 or bystander 26.

Location service 178 may determine the location of computing device(s) 12 and, thereby, the presumed location of patient 4. Location service 178 may use global position system (GPS) data, multilateration, and/or any other known techniques for locating computing devices. In some examples, location service 178 may utilize information from other devices in environment 28 to determine the location of patient 4. For example, location service 178 may use information from any of IMD 10, IoT device(s) 30, or drone 46 to determine the location of patient 4.

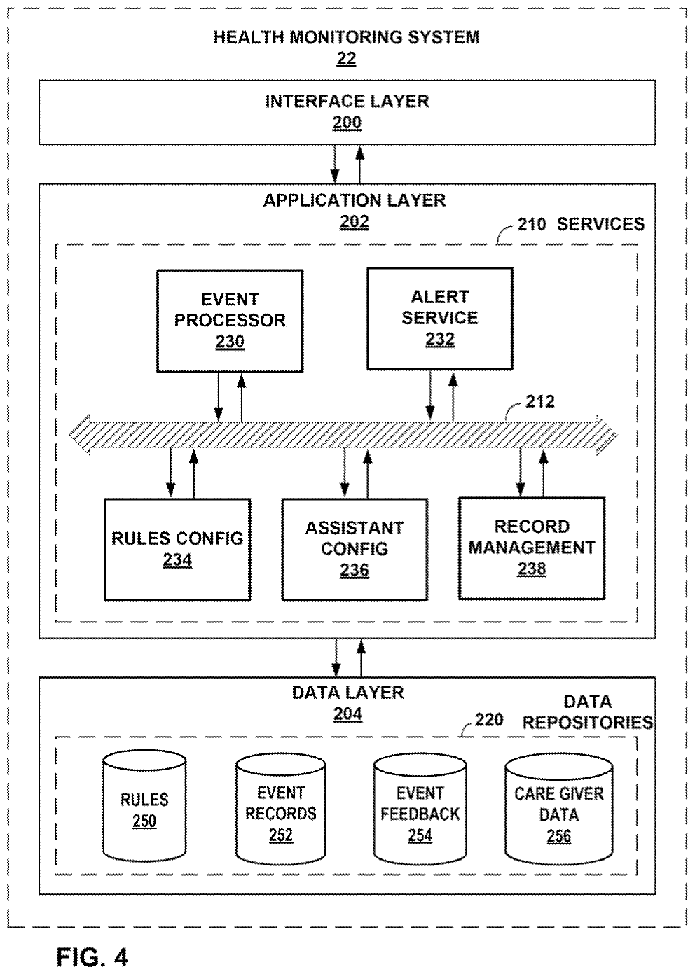

FIG. 4 is a block diagram illustrating an operating perspective of HMS 22. HMS 22 may be implemented in a computing system 20, which may include hardware components such as those of computing device 12, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12, IoT devices 30, computing devices 38, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications, e.g., an alert of an acute health event from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Event processor service 230 may be responsive to receipt of an alert transmission from computing device(s) 12 and/or IoT device(s) 30 indicating that IMB 10 detected an acute health event of patient and, in some examples, that the transmitting device confirmed the detection. Event processor service 230 may initiate performance of any of the operations in response to detection of an acute health event ascribed herein to HMS 22, such as communicating with patient 4, bystander 26, and care providers 40, activating drone 46 and, in some cases, analyzing data to confirm or override the detection of the acute health event by IMD 10.

Record management service 238 may store the patient data included in a received alert message within event records 252. Alert service 232 may package the some or all of the data from the event record, in some cases with additional information as described herein, into one more alert messages for transmission to bystander 26 and/or care providers 40. Care provider data 256 may store data used by alert service 232 to identify to whom to send alerts based on locations of potential bystanders 26 and care providers 40 relative to a location of patient 4 and/or applicability of the care provided by care providers 40 to the acute health event experienced by patient 4.

In examples in which HMS 22 performs an analysis to confirm or override the detection of the acute health event by IMD 10, event processor service 230 may apply one or more rules 250 to the data received in the alert message, e.g., to feature vectors derived by event processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to confirm acute health event detection, (or in examples in which HMS 22 does not confirm event detection) rules 250 maintained by HMS 22 may include rules 196 utilized by computing devices 12 and rules 84 used by IMB 10. In such examples, rules configuration service 234 may be configured to develop and maintain rules 196 and rules 84. Rules configuration service 234 may be configured to modify these rules based on event feedback data 254 that indicates whether the detections and confirmations of acute health events by IMB 10, computing device 12, and/or HMS 22 were accurate. Event feedback data 254 may be received from patient 4, e.g., via computing device(s) 12, or from care providers 40 and/or EHR 24. In some examples, rules configuration service 234 may utilize event records from true and false detections (as indicated by event feedback data 254) and confirmations for supervised machine learning to further train models included as part of rules 250.

As illustrated in the example of FIG. 4, services 210 may also include an assistant configuration service 236 for configuring and interacting with event assistant 176 implemented in computing device 12 or other computing devices.

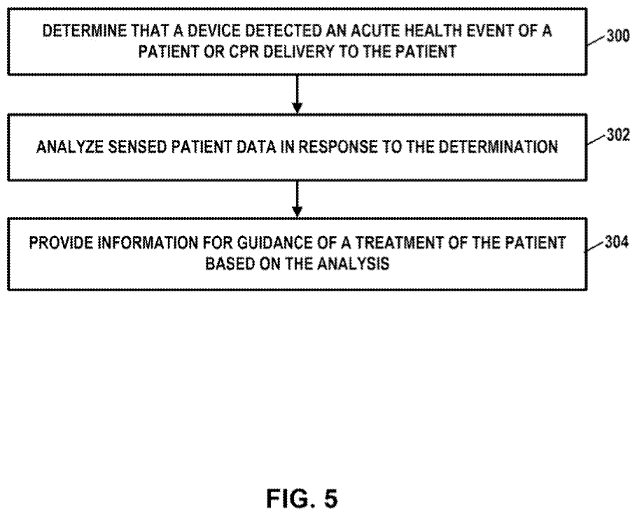

FIG. 5 is a flow diagram illustrating example guidance techniques of this disclosure. processing circuitry 50 or processing circuitry 130 may determine that a device detected an acute health event of patient 4 or delivery of CPR to patient 4 (300). For example, IMD 10 may sense physiological data which may be indicative of an acute health event or the delivery of CPR. Processing circuitry 50 or processing circuitry 130 may determine that IMD 10 detected the acute health event or the delivery of CPR based on the sensed physiological data.

Processing circuitry 50 or processing circuitry 130 may analyze sensed patient data in response to the determination (302). For example, processing circuitry 50 or processing circuitry 130 may analyzed sensed patient data from IMD 10, IoT device(s) 30, or drone 46.

Processing circuitry 50 or processing circuitry 130 may provide information for guidance of a treatment of patient 4 based on the analysis (304). For example, processing circuitry 50 or processing circuitry 130 may provide information for guidance of the treatment of patient 4 to bystander 26, to any of IoT devices 30 (e.g., for providing guidance to bystander 26 or EMS personnel in environment 28), to computing device 42 (e.g., for providing guidance to bystander 26), to any of computing devices 38 (e.g., for providing guidance to respective care providers 40), or to automated CPR device 48.

In some examples, the method of FIG. 5 is performed by IMD 10, a smart phone, a wearable device (e.g., a smart watch), a tablet, a smart TV, a smart speaker, a smart camera, or an IoT device. In some examples, the acute health event is a cardiac event. For example, the acute health event may be sudden cardiac arrest, acute myocardial infarction, lethal arrythmia, or other cardiac event. In some examples, the acute health event may be a non-cardiac event. For example, the acute health event may be a stroke, a fall, respiratory failure, or other non-cardiac event.

In some examples, the information for guidance of the treatment of patient 4 includes first instructions, to a user, on performing CPR. In some examples, processing circuitry 50 or processing circuitry 130 may determine that IMD 10 detected delivery of CPR to patient 4, analyze the delivery of the CPR to patient 4, and provide second instructions indicative of a change to be made to the delivery of the CPR based on the analysis of the delivery of the CPR. In some examples, the second instructions include one of: user instructions for a user (e.g., bystander 26) to change at least one of an intensity or frequency of chest compressions of the CPR, or control instructions configured to control automated CPR device 48 to change at least one of an intensity or frequency of chest compressions of the CPR.

In some examples, analyzing the delivery of the CPR to patient 4 includes analyzing at least one accelerometer signal or ECG signal (e.g., analyzing motion artifacts in the ECG signal). In some examples, at least one of the first instructions or the second instructions comprise at least one of audio instructions or visual instructions. In some examples, the information comprises an ECG, and wherein the ECG is automatically recorded, by the implanted medical device, in response to the detection of the acute health event in patient 4. In some examples, processing circuitry 50 or processing circuitry 130 may classify the ECG, wherein the information includes the classification of the ECG. For example, processing circuitry 50 or processing circuitry 130 may analyze artifacts in the ECG to determine whether bystander 26 should change the CPR, for example to go faster or slower to change the depth of the CPR. In some examples, IMD 10, computing device(s) 12, IoT device(s) 30, or computing device 42 may communicate instructions (e.g., visually or auditorily) to bystander 26 on how to change the delivery of CPR. In some examples, processing circuitry 50 or processing circuitry 130 may determine that the delivery of CPR is not necessary and IMD 10, computing device(s) 12, IoT device(s) 30, or computing device 42 may communicate instructions to bystander 26 to pause the delivery of CPR.

In some examples, providing information for guidance of a treatment of patient 4 includes providing guidance to a care provider 40. In some examples, the guidance includes an automated diagnosis. In some examples, the acute health event includes at least one of sudden cardiac arrest, acute myocardial infarction, lethal arrythmia, stroke, or respiratory failure (e.g., drowning, suffocation, smoke inhalation, etc.).

In some examples, the guidance includes an automated medical diagnosis, and processing circuitry 50 or processing circuitry 130 may determine a location of patient 4, and based on the location of patient 4 and the automated medical diagnosis, determine a medical facility for treatment of patient 4 from among a plurality of medical facilities. In some examples, processing circuitry 50 or processing circuitry 130 may determine a time of a last known well state of patient 4, wherein the determination of the medical facility is further based on the time of the last known well state. In some examples, the determination of the medical facility is further based on equipment located at the medical facility.

Example 1. A method comprising: determining, by processing circuitry, that a device detected an acute health event of a patient or cardiopulmonary resuscitation (CPR) to the patient; analyzing, by the processing circuitry, sensed patient data in response to the determination; and providing, by the processing circuitry, information for guidance of a treatment of the patient based on the analysis.

Example 2. The method of Example 1, wherein the method is performed by an implantable medical device, a smart phone, a wearable device, a tablet, a smart TV, a smart speaker, a smart camera, or an Internet of Things device.

Example 3. The method of Example 1 or Example 2, wherein the acute health event is a cardiac event.

Example 4. The method of Example 1 or Example 2, wherein the acute health event is a non-cardiac event.

Example 5. The method of Example 3 or Example 4, wherein the information for guidance of the treatment of the patient comprises first instructions, to a user, on performing CPR or delivering medication.

Example 6. The method of any of Examples 3-5, further comprising: determining, by the processing circuitry, that the device detected delivery of CPR to the patient; analyzing, by the processing circuitry, the delivery of the CPR to the patient; and providing, by the processing circuitry, second instructions indicative of a change to be made to the delivery of the CPR based on the analysis of the delivery of the CPR.

Example 7. The method of Example 6, wherein the second instructions comprise one of: user instructions for a user to change at least one of an intensity or frequency of chest compressions of the CPR; or control instructions configured to control an automated CPR device to change at least one of an intensity or frequency of chest compressions of the CPR.

Example 8. The method of Example 6 or Example 7, wherein analyzing the delivery of the CPR to the patient comprises analyzing at least one accelerometer signal or an electrocardiogram.

Example 9. The method of any combination of Examples 5-8, wherein at least one of the first instructions or the second instructions comprise at least one of audio instructions or visual instructions.

Example 10. The method of any combination of Examples 5-9, wherein the information comprises an electrocardiogram (ECG), and wherein the ECG is automatically recorded, by the device, in response to the detection of the acute health event in the patient.

Example 11. The method of Example 10, further comprising: classifying, by the processing circuitry, the ECG, wherein the information comprises the classification of the ECG.

Example 12. The method of any combination of Examples 5-11, wherein providing information for guidance of a treatment of the patient comprises providing guidance to a care provider.

Example 13. The method of Example 12, wherein the guidance comprises an automated diagnosis.

Example 14. The method of any combination of Examples 5-13, wherein the acute health event comprises at least one of sudden cardiac arrest, acute myocardial infarction, lethal arrythmia, stroke, or respiratory failure.

Example 15. The method of any combination of Examples 5-14, wherein the guidance comprises an automated medical diagnosis, the method further comprising: determining, by the processing circuitry, a location of the patient; and based on the location of the patient and the automated medical diagnosis, determining, by the processing circuitry, a medical facility for treatment of the patient from among a plurality of medical facilities.

Example 16. The method of Example 15, further comprising: determining, by the processing circuitry, a time of a last known well state of the patient, wherein the determination of the medical facility is further based on the time of the last known well state.

Example 17. The method of Example 15 or Example 16, wherein the determination of the medical facility is further based on equipment located at the medical facility.

Example 18. A device comprising: processing circuitry; and memory comprising instructions that, when executed by the processing circuitry, cause the processing circuitry to: determine that a device detected an acute health event of a patient or delivery of cardiopulmonary resuscitation (CPR) to the patient; analyze sensed patient data in response to the determination; and provide information for guidance of a treatment of the patient based on the analysis.

Example 19. The device of Example 18, wherein the device comprises an implanted medical device, a smart phone, a wearable device, a tablet, a smart TV, a smart speaker, a smart camera, or an Internet of Things device.

Example 20. The device of Example 18 or Example 19, wherein the acute health event is a cardiac event.

Example 21. The device of Example 18 or Example 19, wherein the acute health event is a non-cardiac event.

Example 22. The device of Example 20 or Example 21, wherein the information for guidance of the treatment of the patient comprises first instructions, to a user, on performing CPR or delivering medication.

Example 23. The device of any of Examples 20-22, wherein the instructions further cause processing circuitry to: determine that the device detected delivery of CPR to the patient; analyze the delivery of the CPR to the patient; and provide second instructions indicative of a change to be made to the delivery of the CPR based on the analysis of the delivery of the CPR.

Example 24. The device of Example 23, wherein the second instructions comprise one of: user instructions for a user to change at least one of an intensity or frequency of chest compressions of the CPR; or control instructions configured to control an automated CPR device to change at least one of an intensity or frequency of chest compressions of the CPR.

Example 25. The device of Example 23 or Example 24, wherein as part of analyzing the delivery of the CPR to the patient, the instructions cause the processing circuitry to analyze at least one accelerometer signal or an electrocardiogram.

Example 26. The device of any combination of Examples 22-25, wherein at least one of the first instructions or the second instructions comprise at least one of audio instructions or visual instructions.

Example 27. The device of any combination of Examples 22-26, wherein the information comprises an electrocardiogram (ECG), and wherein the ECG is automatically recorded, by the device, in response to the detection of the acute health event in the patient.

Example 28. The device of Example 27, wherein the instructions further cause the processing circuitry to: classify the ECG, wherein the information comprises the classification of the ECG.

Example 29. The device of any combination of Examples 22-28, wherein as part of providing information for guidance of a treatment of the patient, the instructions cause the processing circuitry to provide guidance to a care provider.

Example 30. The device of Example 29, wherein the guidance comprises an automated diagnosis.

Example 31. The device of any combination of Examples 22-30, wherein the acute health event comprises at least one of sudden cardiac arrest, acute myocardial infarction, lethal arrythmia, stroke, or respiratory failure.

Example 32. The device of any combination of Examples 22-31, wherein the guidance comprises an automated medical diagnosis, and wherein the instructions further cause the processing circuitry to: determine a location of the patient; and based on the location of the patient and the automated medical diagnosis, determine a medical facility for treatment of the patient from among a plurality of medical facilities.

Example 33. The method of Example 32, wherein the instructions further cause the processing circuitry to: determine a time of a last known well state of the patient, wherein the determination of the medical facility is further based on the time of the last known well state.

Example 34. The device of Example 32 or Example 33, wherein the determination of the medical facility is further based on equipment located at the medical facility.

Example 35. A non-transitory computer-readable storage medium storing instructions that, when executed, cause processing circuitry to: determine that a device detected an acute health event of a patient or delivery of cardiopulmonary resuscitation (CPR) to the patient; analyze sensed patient data in response to the determination; and provide information for guidance of a treatment of the patient based on the analysis.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

Various examples have been described. These and other examples are within the scope of the following claims.

1-13. (canceled)

14: A system comprising:
processing circuitry; and
memory comprising instructions that, when executed by the processing circuitry, cause the processing circuitry to:
determine that a device detected an acute health event of a patient or delivery of cardiopulmonary resuscitation (CPR) to the patient;
analyze sensed patient data in response to the determination; and
provide information for guidance of a treatment of the patient based on the analysis.

15: A non-transitory computer-readable storage medium storing instructions that, when executed, cause processing circuitry to:
determine that a device detected an acute health event of a patient or delivery of cardiopulmonary resuscitation (CPR) to the patient;
analyze sensed patient data in response to the determination; and
provide information for guidance of a treatment of the patient based on the analysis.

16: The system of claim 14, comprising an implantable medical device, a smart phone, a wearable device, a tablet, a smart TV, a smart speaker, a smart camera, or an Internet of Things device.

17: The system of claim 14, wherein the acute health event is a non-cardiac event.

18: The system of claim 14, wherein the acute health event is a cardiac event.

19: The system of claim 18, wherein the information for guidance of the treatment of the patient comprises first instructions, to a user, on performing CPR or delivering medication.

20: The system of claim 18, wherein the processing circuitry is further configured to:
determine that the device detected delivery of CPR to the patient;
analyze the delivery of the CPR to the patient; and
provide second instructions indicative of a change to be made to the delivery of the CPR based on the analysis of the delivery of the CPR.

21: The system of claim 20, wherein the second instructions comprise one of:
user instructions for a user to change at least one of an intensity or frequency of chest compressions of the CPR; or
control instructions configured to control an automated CPR device to change at least one of an intensity or frequency of chest compressions of the CPR.

22: The system of claim 20, wherein as part of analyzing the delivery of the CPR to the patient, the processing circuitry is configured to analyze at least one accelerometer signal or an electrocardiogram.

23: The system of claim 18, wherein at least one of the first instructions or the second instructions comprise at least one of audio instructions or visual instructions.

24: The system of claim 18, wherein the information comprises an electrocardiogram (ECG), and wherein the processing circuitry is further configured to automatically record the ECG in response to the detection of the acute health event in the patient.

25: The system of claim 24, wherein the processing circuitry is further configured to classify the ECG, wherein the information comprises the classification of the ECG.

26: The system of claim 18, wherein as part of providing information for guidance of the treatment of the patient, the processing circuitry is configured to provide guidance to a care provider.

27: The system of claim 26, wherein the guidance comprises an automated diagnosis.

28: An insertable cardiac monitor comprising:
a housing configured to be subcutaneously inserted into a patient, the housing comprising a cover;
a plurality of electrodes, at least one of the plurality of electrodes being disposed on a proximal portion of the cover and at least another one of the plurality of electrodes being disposed on a distal portion of the cover;
sensing circuitry configured to sense at least one electrical signal indicative of an occurrence of at least one of an acute health event of a patient or delivery of cardiopulmonary resuscitation (CPR) to the patient via the plurality of electrodes;
processing circuitry; and
memory comprising instructions that, when executed by the processing circuitry, cause the processing circuitry to:
determine, based on the at least one electrical signal, the occurrence of the acute health event of the patient or the delivery of the CPR to the patient;
analyze sensed patient data in response to the determination; and
provide information for guidance of a treatment of the patient based on the analysis.

29: The insertable cardiac monitor of claim 28, wherein the acute health event is a non-cardiac event.

30: The insertable cardiac monitor of claim 28, wherein the acute health event is a cardiac event.

31: The insertable cardiac monitor of claim 30, wherein the information for guidance of the treatment of the patient comprises first instructions, to a user, on performing CPR or delivering medication.

32: The insertable cardiac monitor of claim 30, wherein the processing circuitry is further configured to:
detected delivery of CPR to the patient;
analyze the delivery of the CPR to the patient; and
provide second instructions indicative of a change to be made to the delivery of the CPR based on the analysis of the delivery of the CPR.

33: The insertable cardiac monitor of claim 32, wherein the second instructions comprise one of:
user instructions for a user to change at least one of an intensity or frequency of chest compressions of the CPR; or
control instructions configured to control an automated CPR device to change at least one of an intensity or frequency of chest compressions of the CPR.
